(19) United States
(12) Patent Application Publication
(10) Pub. No.: US 2023/0166848 A1
(43) Pub. Date: Jun. 1, 2023

(54) METHOD AND APPARATUSES FOR DELIVERING HYPERBARIC GAS AND/OR TREATING RESPIRATORY ILLNESSES, POST COVID SYNDROME(S) AND CHRONIC TRAUMATIC ENCEPHALOPATHY

(71) Applicant: The Climate Foundation, Seattle, WA (US)

(72) Inventors: Brian VON HERZEN, Minden, NV (US); Daniel REYNOLDS, Royston (GB)

(21) Appl. No.: 18/058,653

(22) Filed: Nov. 23, 2022

Related U.S. Application Data
(63) Continuation of application No. PCT/US2021/033570, filed on May 21, 2021.
(60) Provisional application No. 63/029,416, filed on May 23, 2020.

(51) Int. Cl. B64D 13/08; A62B 7/14; A62B 7/06; A62B 18/08; A62B 9/02; A62B 7/10

(52) U.S. Cl. CPC B64D 13/08; A62B 7/14; A62B 7/06; A62B 18/084; A62B 9/02; A62B 7/10; A61M 16/0672

(57) ABSTRACT

An apparatus is disclosed that includes an aircraft or repurposed aircraft or fuselage having a cabin capable of pressurization, a way to pressurize the cabin whether by built in machinery or external apparatus, a way to deliver oxygen to a plurality of persons in the cabin, a source of hyperbaric oxygen, a pressure gauge or regulator configured to measure or regulate a pressure of the hyperbaric oxygen or the aircraft, a plurality of face or head coverings configured to provide the hyperbaric oxygen to persons or patients in need thereof, and an optional exhaust system configured to remove gas(es) from the face or head coverings without releasing the gas(es) into the cabin. Each of the face or head coverings includes a gas inlet, a gas outlet, and one or more seals adapted to contain oxygen in the face or head covering at a pressure greater than 1 atm. A related kit and a related method of treating persons with hyperbaric oxygen are also disclosed.

### RELATED APPLICATION(S)

This application is a continuation of International Pat. Appl. No. PCT/US2021/033570, filed May 21, 2021, pending, which claims priority to U.S. Provisional Pat. Appl. No. 63/029,416, filed on May 23, 2020, both of which are incorporated herein by reference.

### FIELD OF THE INVENTION

The present invention relates to apparatuses for and a method of delivering a hyperbaric gas, such as oxygen or oxygen-rich air, to a person in need thereof.

### DISCUSSION OF THE BACKGROUND

It is believed that the pandemic caused by the SARS-CoV-2 coronavirus is likely to last as long as two years, and that it may not be controlled until about two-thirds of the world's population is immune. It is estimated that over five million people may die in the next 24 months under the current standard of care. Aiming for herd immunity could result, for example, in another 15,000,000 deaths in India alone under present standards.

Vaccines are not expected by vaccine experts to be available to the U.S. general public until mid-2021 at the earliest, and not until early- to mid-2022 globally. In addition, 650 million to 850 million needles and syringes will be needed to administer a vaccine in the US alone. It is estimated that it could take 2 years for current U.S. manufacturing resources to produce that quantity of needles and syringes. For example, the U.S. Federal Government placed first orders for just the first half of the minimum required amount, 320 million syringes, on May 1, 2020. It is not known when the syringes will be available, as supply shortages in the materials for making the syringes are possible.

Many patients with COVID-19 experience oxygenation failure. Many patients coming in to hospitals with COVID-19 symptoms present with hypoxemia. A number of physicians in New York observed that some severe COVID-19 cases are not exhibiting respiratory failure. Instead, they are showing oxygenation failure with silent hypoxia. This is an example of abnormal physiology associated with COVID-19 that the medical community does not yet fully understand.

Approximately 25-30% of incoming patients present with hypoxemia during triage, requiring supplemental oxygen upon admission. COVID-19 is believed to attack red blood cells, which can induce hypoxemia in the bloodstream and hypoxia in tissues. So-called “silent” hypoxia and severe hypoxemia can lead to multiple organ failure due to lack of oxygen. Chronic hypoxia can result in coagulation problems, blockages, host inflammatory syndrome, deep vein thrombosis (DVT), strokes, cascading failures, and death. It is estimated that brain damage in 40% of COVID-19 patients placed on ventilator therapy may be due to low oxygen, blood clots, or both.

Hypoxemia and chronic hypoxia can explain perhaps 80% of the symptoms in late-stage COVID-19 patients described in the previous paragraph. One fast, practical diagnostic tool used in New York City for indications of COVID-19 under scarcity of COVID testing in the emergency room in early 2020 was pulse oximetry. Pulse oximeters are available in most pharmacies for a price of about $40.

A common therapy used to treat acute respiratory distress syndrome (ARDS) in COVID-19 patients is to place the patient on a ventilator. Ventilators increase respiration, but not necessarily oxygenation over pure O₂, as the O₂ partial pressure in the gas supplied to the patient by the ventilator is typically not substantially greater than atmospheric and the fundamental problem is the inability of the patient to absorb sufficient oxygen, due to a lack of sufficient functional area for gas exchange, due to inflammation of the lung surfaces and/or hypoxic vasoconstriction.

Hypoxia causes vasoconstriction of pulmonary blood vessels, exacerbating hypoxemia. Hypoxia is associated with the host inflammatory response phase (phase IIA in the graph shown in FIG. 1). COVID-19 is detectable in Phase IIA (FIG. 1) before the host inflammatory cascade. Thromboinflammation (a possible consequence of the host inflammatory response) relates to coagulation, DVT, strokes and blockages. Both Type L and Type H COVID-19 phenotypes exhibit hypoxemia.

In one study, the median time to dyspnea is 6.5 days after the onset of symptoms; in other studies, the median time to dyspnea ranges from 5 to 8 days. The median time to ARDS in one study was 9 days after the onset of symptoms. The time between dyspnea and ARDS provides a window of about 1-4 days to provide early treatment before the onset of ARDS. Ideally, hypoxia problems can be identified early, before chronic hypoxia sets in.

A study in Wuhan, China treated patients with hyperbaric oxygen (HBO) to treat hypoxic vasoconstriction. FIG. 2 shows the results of 5 daily HBO treatments of 90-120 minutes at 1.4-1.6 atmospheres absolute (ATA) to each of 5 COVID-19 patients, 2 of whom were critical, and 3 of whom were severe. The HBO treatments provided an immediate increase in oxygen saturation levels (SO₂) shown in the data represented by squares (the “After HBOT” line in FIG. 2). HBO treatment of these patients was life-saving, according to the physicians who conducted the study in Wuhan.

It is believed that hyperbaric oxygen therapy (HBOT) has been shown to increase tissue oxygenation in the brain, improve neuroplasticity and revitalize the cognitive functions that have been chronically damaged in chronic traumatic encephalopathy (CTE). Some patients experienced increased blood flow in the brain and significant improvements in cognitive and motor functions, including reaction time, memory, and reasoning. It has also been reported that HBOT increased visual motor speed and reaction time, and reduced frequency and severity of headaches, in at least one CTE patient.

Globally only 4500 hyperbaric oxygen chambers are available. Most of these chambers are designed for only 1 or 2 patients. Furthermore, when used to treat a COVID-19 patient, the hyperbaric chamber requires a thorough, difficult and time-consuming cleaning, exposing medical staff to potential infection, given that the SARS-CoV-2 virus can apparently live for quite a while as an aerosol in a hyperbaric oxygen chamber or cabin.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

It is believed that early measurement of oxygen saturation level enables preventative therapy with better outcomes. Hyperbaric oxygen (HBO) therapy (HBOT) solves hypoxia directly, delivering up to ten times the partial pressure of oxygen available in atmospheric air, and up to 100% more oxygen than can be delivered by ventilators or a continuous positive airway pressure (CPAP) machine. Many HBO systems can deliver up to 2 bars (atm) of oxygen, providing 10 times the oxygen of air at standard temperature and pressure (STP), and 100% more oxygen than a ventilator providing pure oxygen to a patient.

Hyperbaric oxygen therapy has been shown to provide antimicrobial activity for infections. Hyperbaric oxygen therapy has also been shown to reduce or prevent coagulation disorders in an experimental model of multiple organ failure syndrome.

The present invention solves the problem of the limited number of available hyperbaric chambers by using existing equipment with minimal modification. The cabins of commercial aircraft can be pressurized to levels equivalent to those of many hyperbaric chambers, and commercial aircraft are configured to provide emergency oxygen at ambient pressures to passengers (typically in the event of a loss of pressure in the cabin or other emergency). Such aircraft can serve as mobile units that can go to locations (e.g., cities, counties, states, provinces, etc.) where needed. They are widely available around the world, and provide a potential capacity to treat up to 2 million patients concurrently.

Acute COVID-19 infection is associated with inflammation of the lungs, impeding gas exchange and reducing the available surface area of the lung. Furthermore, when part of a patient's lung is damaged, it may become hypoxic. When lung tissue becomes hypoxic, it vasoconstricts the blood vessels that are in the damaged part of the lung, and shunts more blood to the healthy part of the lung. One issue with COVID-19 is that it does not necessarily affect only part of the lung. In at least some cases, it appears that the patient's entire lungs experience hypoxemia. This can lead to vasoconstriction in the pulmonary blood vessels. This results in reduced oxygenation of the blood. It also means that once a patient is in a chronic hypoxic condition, it is difficult for the patient to recover. Hyperbaric oxygen therapy (e.g., at 8 to 10 times the concentration of atmospheric oxygen in the atmosphere at STP) overcomes the hypoxic condition in the patient's lungs and is believed to relieve vasoconstriction in the lungs, thereby enabling the patient to breathe relatively comfortably during the HBOT and recover from the hypoxemia.

Using commercial or other pressurized aircraft as hyperbaric chambers enables providing relatively simple, noninvasive respiratory support and/or therapy to a much larger number of patients than can existing hyperbaric chambers, as well as an early treatment option that can keep people out of hospitals and off ventilators. Mobile hospital aircraft can also be relocated to serve global epidemics in any major location.

For every 100 widebody aircraft grounded (at ˜$50M per aircraft), the economy loses $5B in stranded assets. Putting these stranded assets to use not only stems the flow of losses due to grounding the aircraft, but could result in revenues to the airlines offering their aircraft for such a use. It also makes possible employment to the flight crews who can ensure patient and medical/maintenance staff safety on the ground during pressurization and other operations.

Part of the reason for the belief that early measurement of oxygen saturation level enables preventative therapy with better outcomes is that hypoxic vasoconstriction of pulmonary blood vessels may represent a kind of “runaway” feedback loop that makes a bad problem worse under COVID-19. HBOT may reverse hypoxic vasoconstriction, halting acute hypoxemia and providing the patient's organs, including pulmonary blood vessels, with rest and an oxygenated reset, enabling a reduction in hypoxemia lasting as long as 24 hours (or more), at which point the patient may participate in another HBO session. Data from small trials in Louisiana and Wuhan showed that patients were able to avoid mechanical ventilation and recover from serious COVID-19 illness using HBOT.

Thus, an aspect of the present invention relates to an apparatus for providing hyperbaric oxygen to patients in need thereof, comprising an aircraft with a cabin capable of pressurization, a source of oxygen, a pressure gauge and regulator configured to measure and regulate the supply of oxygen, a plurality of nasal cannulas or face or head coverings, and an exhaust system. The aircraft has a cabin and an emergency air or oxygen delivery system configured to deliver air or oxygen to a plurality of persons in the cabin. The nasal cannulas or face or head coverings are configured to provide the hyperbaric oxygen to the patients. Each of the face or head coverings includes a gas inlet, a gas outlet, and one or more seals adapted to contain oxygen in the nasal cannula or face or head covering at a pressure greater than 1 atm. The exhaust system is configured to remove gas(es) from the face or head coverings without releasing the gas(es) into the cabin.

In some embodiments, the source of hyperbaric oxygen comprises liquid oxygen in a container configured to store liquid oxygen therein, such as a Dewar vessel (a “Dewar”). In further embodiments, the apparatus further comprises a heater in the container, and a controller configured to receive a pressure of the hyperbaric oxygen from the pressure gauge or regulator. The heater is configured to add thermal energy to the liquid oxygen. When the pressure of the hyperbaric oxygen is below a predetermined threshold pressure, the controller controls the amount of thermal energy added to the liquid oxygen to increase the pressure of the hyperbaric oxygen to a value greater than the predetermined threshold pressure.

In alternative embodiments, the source of hyperbaric oxygen comprises a plurality of tanks of oxygen operably connected to the pressure gauge or regulator. The oxygen tanks may be stored or located in the cabin, external to the aircraft, or in a cargo hold of the aircraft.

In various embodiments, each of the face or head coverings comprises a flexible, at least partially transparent head covering configured to cover the entire head of a patient. Alternatively, each of the face or head coverings may comprise a stiff, optionally spherical head covering configured to cover the entire head of the patient. The stiff head covering includes an opening through which the patient's head is inserted. In such head coverings, the seal may comprise an elastic fitting or band, configured to secure the head covering to the head of the patient (e.g., around the neck) in a substantially airtight manner, but not so tight that the patient has difficulty breathing. The elastic fitting or band may allow for some limited flow of the hyperbaric oxygen out of the head covering. For example, the elastic fitting or band may be covered with a loose cloth or fabric covering to increase comfort and facilitate easy breathing, without sacrificing much or any of the sealing properties of the elastic fitting or band.

In further alternatives, the face or head coverings comprises a nose-and-mouth covering (a “mask”), configured to provide the hyperbaric oxygen to the patient. In such masks, the seal may comprise an outermost rubber, silicone or other polymeric layer configured to contact the patient's face and, to the extent the mask extends to the patient's neck area, the patient's neck. The mask may be secured to the patient's face, head and/or neck with one or more rubber, silicone or elastic bands or straps. Thus, each of the face or head coverings comprises an elastic fitting or band, configured to secure the face or head covering to the face or head of the patient in a substantially airtight manner.

In some embodiments, the apparatus may further comprise a supply tube, hose or conduit connected between the emergency air or oxygen delivery system and the gas inlet of the face or head covering. In some cases, the supply tube, hose or conduit may have a first end connected to an outlet of the emergency air or oxygen delivery system (e.g., over the patient's seat, where the emergency oxygen mask is located in a conventional passenger aircraft) and a second end connected to the gas inlet of the face or head covering. The supply tube, hose or conduit (or the first and/or second ends thereof) may have a first connector and/or a second connector adapted to connect the supply tube, hose or conduit to outlet of the emergency air or oxygen delivery system or the gas inlet of the face or head covering, respectively. In other cases, the supply tube, hose or conduit may include (i) a first tube connected to or integrated with the emergency air or oxygen delivery system and (ii) a second tube connected to or integrated with the gas inlet of the face or head covering. The first and second tubes may connect to each other through a supply tube connector, which may be separate from or integrated with one of the first and second tubes.

The cabin generally has a wall, a floor, and a ceiling, and the aircraft generally has an exterior shell or fuselage. In such cases, the exhaust system may comprise (i) one or more (e.g., a plurality of) exhaust lines and/or an exhaust manifold under the cabin floor, above the cabin ceiling, or between the cabin wall and the exterior shell or fuselage, and/or (ii) a plurality of exhaust tubes, hoses or conduits, each connected between a unique gas outlet (on a face or head covering) and the cabin floor, cabin ceiling, or cabin wall. Alternatively, a return air grill in a conventional cabin air recirculation system, under or over the cabin in a commercial passenger aircraft, may be replaced with a panel and connectors thereon (each receiving an end of one exhaust tube, hose or conduit from the gas outlet) for a row (or section thereof) of seats. The gases thus exhausted from the nasal cannulas or face or head coverings on the patients may then be diverted completely to exit ducts to dispose the gases through the fuselage and outside of the aircraft, rather than sending part of the exhaust gases to the mixing manifold in the conventional cabin air recirculation system. The exhaust gases may be passed through a high efficiency particulate air (HEPA) filter prior to exiting the aircraft. Thus, the exhaust system may further comprise a plurality of wall, floor or ceiling connectors in the cabin wall, configured to connect a corresponding one of the exhaust tubes, hoses or conduits to the exhaust line(s) or the exhaust manifold.

In further alternative embodiments, the present apparatus may further comprise (i) a breathing bag connected to the gas outlet, (ii) a CO₂ scrubber canister configured to remove CO₂ from the air or oxygen to be delivered to one of the patients wearing a corresponding one of the nasal cannulas or face or head coverings, and/or (iii) a hose connecting the CO₂ scrubber canister and the breathing bag. The breathing bag may be operably equipped with (1) a condensation drain valve configured to remove liquid from the breathing bag and/or (2) an automatic overpressure valve configured to allow gas to escape from the breathing bag when the pressure in the breathing bag exceeds a predetermined threshold. The predetermined threshold pressure in the breathing bag may be the same as or slightly less than the target pressure for the air or oxygen in the face or head covering (e.g., at or slightly above the ambient pressure in the cabin, which is in the range of 1.3-2.0 atm, in order to enable positive-pressure breathing if required to improve ventilation).

In another aspect, the present invention relates to a kit for providing hyperbaric oxygen to a plurality of persons in a cabin of an aircraft having an emergency air or oxygen delivery system therein. The kit comprises a pressure gauge or regulator configured to measure or regulate the pressure of the oxygen, a conduit or conduit system configured to transport the oxygen from the regulator to the emergency air or oxygen delivery system, a plurality of face or head coverings configured to provide the oxygen to the persons, a plurality of supply tubes or hoses, and a plurality of exhaust tubes or hoses. Each of the face or head coverings includes a gas inlet, a gas outlet, and one or more seals adapted to contain oxygen in the face or head covering at a pressure equal to or slightly greater than ambient pressure (e.g., ≥1 atm, such as 1.4-2.0 atm). Each of the supply tubes or hoses is configured to transport the oxygen from the emergency air or oxygen delivery system to a unique one of the gas inlets. Each exhaust tube or hose is configured to transport gas(es) from a unique one of the gas outlets to an exhaust system in the aircraft.

Similar to the present apparatus, the face or head coverings may comprise an elastic fitting or band, configured to secure the face or head covering to a face or head of one of the persons in a substantially airtight manner. In different embodiments, the face or head covering may comprise (i) a mask with a sealing layer adapted to contact the face (and optionally the neck) of the person, (ii) a flexible, at least partially transparent head covering configured to cover the entire head of the person, or (iii) a stiff, spherical head covering configured to cover the entire head of the person. In the case of the stiff, spherical head covering, it may include an opening through which the person's head is inserted.

Other embodiments of the kit may include one or more components or structures useful for the present apparatus, other than components or structures that are part of the conventional aircraft.

A further aspect of the present invention relates to a method of treating a plurality of patients with hyperbaric oxygen, comprising delivering the oxygen to an emergency air or oxygen delivery system in an aircraft, placing a face or head covering on or over the face or head of each of the patients, transporting the oxygen to the plurality of face or head coverings using the emergency air or oxygen delivery system, allowing the patients to breathe the oxygen in the face or head covering for a length of time sufficient to improve an average or median oxygen saturation level of the patients, and exhausting gases from each of the face or head coverings using an exhaust system in the aircraft. The face or head covering is configured to provide the oxygen to the patient, and it has one or more seals adapted to contain oxygen in the face or head covering at a pressure greater than 1 atm (capable of sealing at around 0.03 atm above ambient). In most embodiments, the length of time is at least 30 minutes (or any length of time or ranges of time lengths of at least 30 minutes; e.g., from 1 to 8 hours, 90 minutes to 4 hours, etc.).

In some embodiments, the method further comprises regulating or controlling a pressure of the oxygen from a source of the oxygen as the oxygen is delivered to the emergency air or oxygen delivery system. In other or further embodiments, the source of the hyperbaric oxygen comprises liquid oxygen in a container configured to store liquid oxygen therein, similar to the present apparatus. In such embodiments, the method may further comprise adding thermal energy to (e.g., heating) the liquid oxygen to evaporate it. In alternative embodiments, the source of the oxygen comprises a plurality of tanks of oxygen, similar to the present apparatus.

In some embodiments, placing the face or head covering on or over the face or head of the patients comprises placing the head covering over the head of each of the patients. Similar to the present apparatus and kit, the head covering may comprise an elastic fitting or band, configured to secure the head covering to the head of one of the patients in a substantially airtight manner. In further embodiments, the head covering may comprise (i) a flexible, at least partially transparent head covering configured to cover the entire head of the patient, or (ii) a stiff, spherical head covering configured to cover the entire head of the patient. In the latter case, the stiff, spherical head covering includes an opening, and placing the head covering over the head of each patient comprises inserting the patient's head through the opening.

Similar to the present apparatus and kit, various embodiments of exhausting the gases may comprise (i) connecting an exhaust tube, hose or conduit between the face or head covering and the exhaust system in the aircraft, (ii) pulling the gases from the face or head covering using a fan or a vacuum, (iii) passing the gases through a filter (e.g., prior to venting or exhausting the gases outside of the aircraft), and/or (iv) venting or exhausting the gases outside of the aircraft. Each of the various embodiments of exhausting the gases may include further details as described herein for the present apparatus and/or kit.

Yet another aspect of the invention relates to a rebreather apparatus, comprising a face or head covering configured to provide hyperbaric air or oxygen to a patient in need thereof, a breathing bag operably equipped with (i) a condensation drain valve configured to remove liquid from the breathing bag and (ii) an automatic overpressure valve configured to allow gas to escape from the breathing bag when a pressure in the breathing bag exceeds a predetermined threshold, a CO₂ scrubber canister configured to remove CO₂ from air or oxygen in the apparatus, a hose connecting the CO₂ scrubber canister and the breathing bag, and an oxygen supply operably connected to the hose, configured to provide the hyperbaric air or oxygen. The face or head covering includes a gas inlet, a gas outlet, and one or more seals adapted to contain the air or oxygen in the face or head covering at a pressure equal to or slightly greater than ambient pressure (e.g., to facilitate a positive pressure to be maintained in the lungs if needed).

In some embodiments, the rebreather apparatus may further comprise (i) a first one-way valve between the gas outlet and the breathing bag, and (ii) a second one-way valve between the CO₂ scrubber canister and the gas inlet. In other or further embodiments, the face or head covering may comprise (i) a flexible, at least partially transparent head covering configured to cover the entire head of the patient and (ii) a replaceable latex or silicone neck seal or ring.

In some embodiments, the CO₂ scrubber canister may comprise a housing, CO₂ absorbent material within the housing, a grid upstream from the CO₂ absorbent material, configured to retain the CO₂ absorbent material in the housing, and a dust filter and grid downstream from the CO₂ absorbent material. In other or further embodiments, the oxygen supply may comprise an oxygen bottle, cylinder or tank, an on-off valve configured to open and close the oxygen bottle, cylinder or tank, and a regulator (e.g., a pressure regulator) configured to control a flow of oxygen from the oxygen bottle, cylinder or tank (e.g., to match the patient's or user's metabolic needs). In such other or further embodiments, the rebreather apparatus may further comprise a needle valve configured to control the flow of the oxygen from the oxygen supply to the hose.

The present invention uses either functional or stranded/grounded aircraft for affordable hyperbaric oxygen (HBO) treatment to supplement and enhance infrastructure for addressing COVID-19 symptoms before hospitalization. The use of airplanes for HBO treatment allows for an unprecedented capacity for concurrently treating up to 200 patients (or more, in some cases) per airplane. The invention can prevent hospitalization for some patients, resulting in reduced critical resource demand, in particular preventing patients from declining to the point of needing intubation and long-term ventilation.

An HBO-retrofitted airplane augments community resilience through increased available and affordable treatment to vulnerable populations. The invention directly benefits those with higher risk of negative outcomes due to age, pre-existing conditions, and medical history, as well as those facing financial limitations/difficulties after the crisis, including the unemployed. In addition, the present invention enables the return to work of airline employees, as operation of the grounded airplane to provide HBO therapy to patients requires ground crews, pilots, and aircraft staff.

The present invention reduces, and has the potential to eliminate most of, the risk of fatality due to COVID-19, and may be more effective treatment in at least some cases than that provided by ventilators and CPAP machines. The present invention may also be useful in treating CTE patients. It also has the capacity to provide millions of treatments over a relatively short time frame (e.g., before a vaccine becomes widely available). HBO is also being shown to be effective in helping persons recovering from brain fog and other neurological, cardiovascular and pulmonary symptoms of post-COVID syndrome affecting millions across the world. It also is effective in accelerating recovery from sports injuries and fatigue. It also is effective in treating forms of diabetes, especially foot injuries, and peripheral vascular disease. Implementation of the present invention is not limited by geographic region, as the modifications for providing HBO therapy do not impact the airplane's mobility. However, pressurizable aircraft are present in substantially every major city in every country on Earth, presenting an opportunity for national and global resilience in the fight against the SARS-CoV-2 virus.

### BRIEF DESCRIPTION OF THE DRAWINGS

FIG. 1 is a diagram showing the phases of a COVID-19 infection, the severity of the illness as a function of time, and some clinical symptoms and signs of the illness.

FIG. 2 is a graph showing the average oxygen saturation level of patients treated daily with hyperbaric oxygen in a study conducted in Wuhan, China.

FIG. 3 is a diagram of a commercial aircraft configured to provide hyperbaric oxygen to people therein, in accordance with one or more embodiments of the present invention.

FIG. 4A is a diagram of patients receiving hyperbaric oxygen in a row of seats in a commercial aircraft equipped with a system and/or apparatus configured to provide hyperbaric oxygen to patients in need thereof, in accordance with one or more embodiments of the present invention.

FIG. 4B is a diagram of rows of seats in a commercial aircraft equipped with a system and/or apparatus configured to provide hyperbaric oxygen to patients in need thereof, in accordance with one or more embodiments of the present invention.

FIG. 5 shows an exemplary head covering or helmet useful in the present apparatus, kit and method, in accordance with one or more embodiments of the present invention.

FIGS. 6A-D show exemplary head coverings/helmets that may be useful in the present apparatus, kit and method, in accordance with embodiments of the present invention.

FIG. 7 shows an exemplary head face shield useful in the present apparatus, kit and method, in accordance with one or more embodiments of the present invention.

FIG. 8 shows an exemplary emergency oxygen rebreather for use in accordance with one or more embodiments of the present invention.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures and components have not been described in detail so as not to unnecessarily obscure aspects of the present invention. Furthermore, it should be understood that the possible permutations and combinations described herein are not meant to limit the invention. Specifically, variations that are not inconsistent may be mixed and matched as desired.

For the sake of convenience and simplicity, the terms “tube,” “hose,” “conduit” and grammatical variations thereof are, in general, interchangeable and may be used interchangeably herein, but are generally given their art-recognized meanings. Wherever one such term is used, it also encompasses the other terms. Similarly, for convenience and simplicity, the terms “hyperbaric oxygen” and “HBO” may be used interchangeably herein, and generally refer to oxygen at a pressure or partial pressure >1 ATA or >1 atm at STP. Wherever one such term is used, it also encompasses the other terms. The terms “saturation pressure of oxygen” and “SPO2” may be used interchangeably as well, but generally refer to the oxygen saturation in a patient's blood, measurable by a noninvasive, over-the-counter pulse oximeter. In addition, for convenience and simplicity, the terms “part,” “portion,” and “region” may be used interchangeably but these terms are also generally given their art-recognized meanings. Also, unless indicated otherwise from the context of its use herein, the terms “known,” “fixed,” “given,” “certain” and “predetermined” generally refer to a value, quantity, parameter, constraint, condition, state, process, procedure, method, practice, or combination thereof that is, in theory, variable, but is typically set in advance and not varied thereafter when in use.

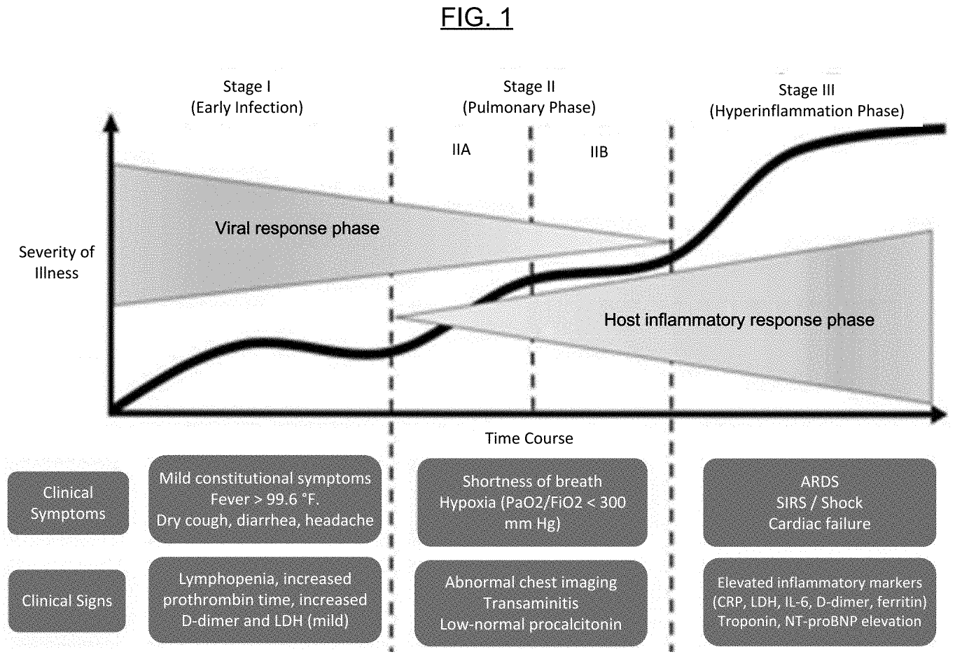

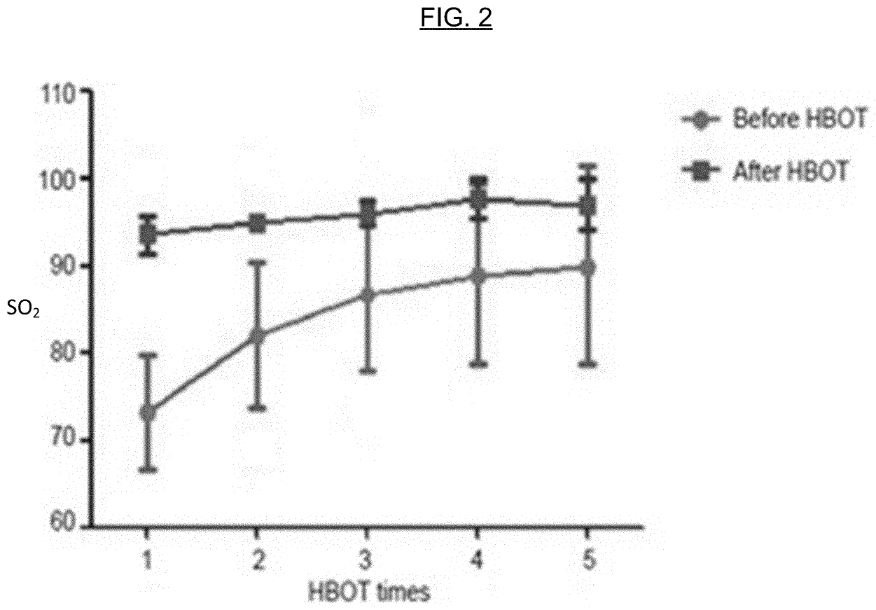

HBO therapy can increase patient oxygen saturation levels over and above ventilators. According to FIG. 1, in typical COVID-19 disease progression, there is a window of about 2.5 days+/−1.5 days after Phase I and during Phase IIA in which hypoxemia is noticeable, but severe ARDS has not yet set in. Even without COVID-19 detection, this hypoxemia can be detected with a pulse oximeter, and a low SPO2 level (e.g., <95%, <93%, etc.) can provide an indication for HBO therapy, before serious ARDS develops. In some cases, such HBO therapy may be able to reverse inflammatory response and other, more serious diseases.

Assuming 8,800 widebody pressurized aircraft are available, in service, and fitted with the present system and apparatus(es) for delivering HBO to a patient in need thereof, a capacity of 1-2M patients concurrently per therapy session can be provided. Assuming a 90-minute session and 30-60 minutes between sessions to allow patients to depart, staff to deep clean and disinfect the plane, medical personnel and attendants to exchange used personal protective equipment for new/clean/sterile equipment, and a new group of patients to enter and be seated (and optionally, be fitted with an HBO breathing apparatus), between 4M and 10M patients can be treated per day, more than meeting the need for such therapy.

An Exemplary Aircraft Configured to Provide Hyperbaric Oxygen Therapy and Exemplary Equipment and Methods for Providing Hyperbaric Oxygen to People in Need Thereof

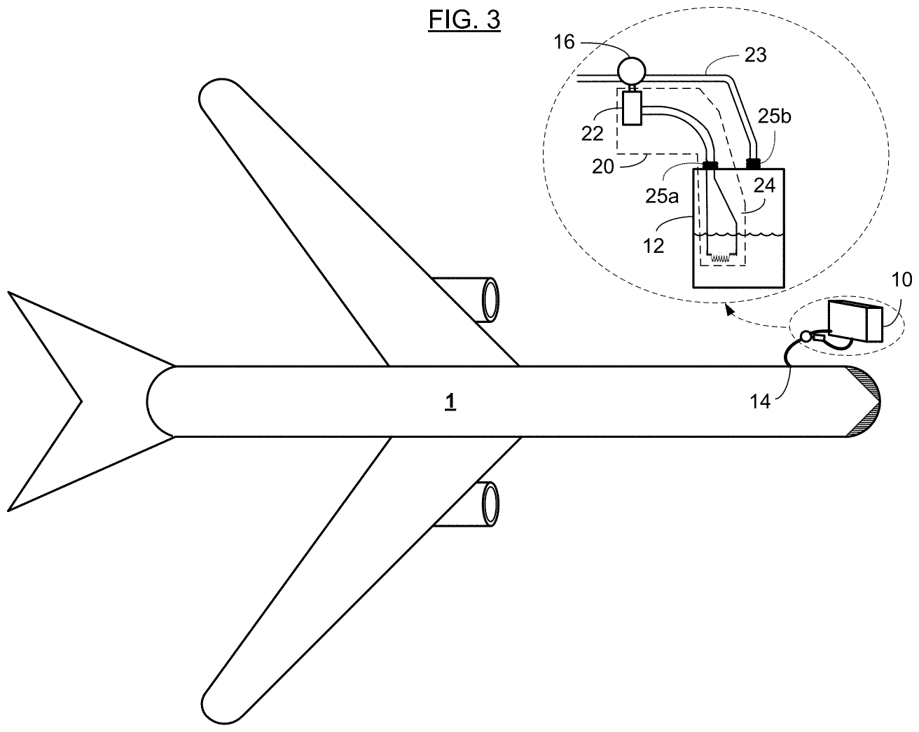

FIG. 3 shows a pressurized aircraft 1 receiving hyperbaric oxygen from an apparatus 10 configured to provide hyperbaric oxygen. The aircraft may be a Boeing 747 or 787 aircraft, and in other advanced aircraft such as the Boeing 777X and at least one Airbus aircraft (e.g., the Airbus A340, A350 and A380 aircraft) or any aircraft capable of pressurization to the required level. Also, the aircraft may be a conventional medical or casualty evacuation (medevac or casevac) aircraft. The apparatus 10 comprises a Dewar or other insulated container 12 capable of storing liquid oxygen, a barostat 16 configured to provide gas-phase oxygen to the airplane at a certain pressure or within a predetermined pressure range, and a heater 20. The heater 20 includes a controller 22 that receives pressure information from the barostat 16 and controls an electrical current to an oxygen-compatible conductive wire (e.g., nickel or a nickel alloy such as Inconel or Monel) or other heating element 24 in the Dewar 12. The heating element 24 may pass through a seal 25a in a cap or lid of the Dewar 12.

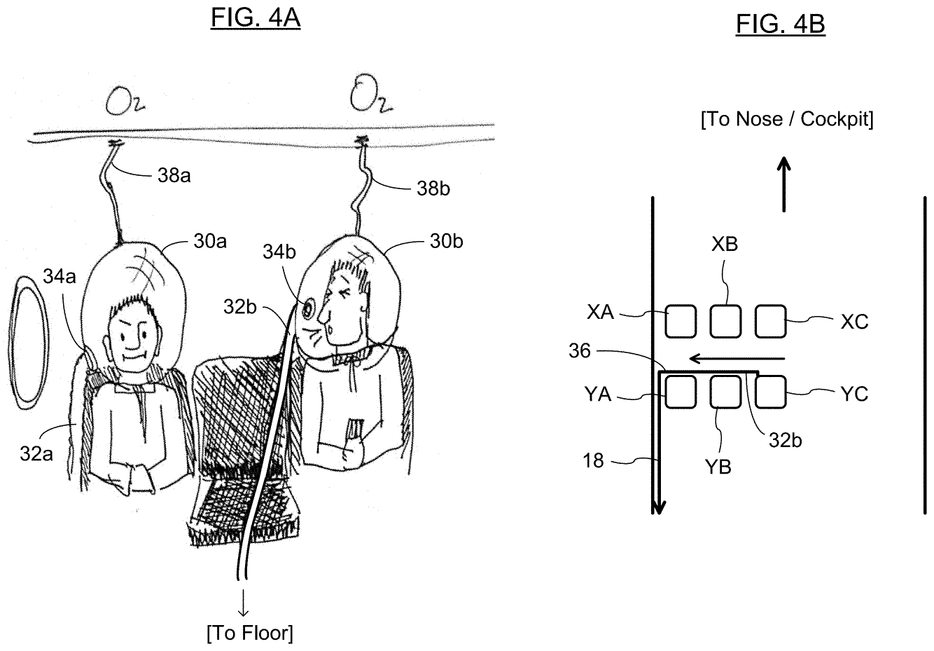

The controllable resistive heater (e.g., controller 22 and heating element 24) controls the amount of liquid oxygen that evaporates in the Dewar 12, and thus, the gas pressure in the Dewar itself and in the tube, hose or conduit 23 connected to the barostat 16 and the Dewar 12 (at a seal or connector 25b in the cap or lid of the Dewar 12). The controller 22 is programmed or otherwise set to control the pressure of the oxygen gas provided to the airplane within a range typically of from 1.4-2.0 ATA (or any value or range of values therein) to the patients (who, as a group, may be fitted with an array of masks or helmets; see, e.g., FIG. 4A), plus enough additional pressure to account for the pressure drop from the Dewar 12 to the emergency oxygen distribution system and the masks/helmets, and to the extent needed or desired, to push exhaust out from the aircraft 1. This target pressure is a function of the ambient pressure of the aircraft cabin. If the Dewar 12 is inside the cabin, then a gauge differential pressure barostat 16 can be used, in which case the pressure regulator (e.g., in the controller 20) may provide only the current necessary to support a gauge pressure sufficient for the HBO flow through the distribution system and through the patient's masks or helmets. For example, the gauge pressure of the barostat 16 may be set at 10-30 psi (e.g., about 0.7-2.0 atm), although it is not limited to this range. When the gauge pressure of the barostat 16 is differential, it may be set in the lower end of the range (e.g., 0.6-1.2 atm), and when the gauge pressure of the barostat 16 is absolute, it may be set in the higher end of the range (e.g., 1.6-2.2 atm).

The controller 22 receives the actual pressure of the oxygen flowing to the airplane from the barostat 16, and when the pressure drops below the minimum setting in the controller 22, the controller 22 passes current through the heating element 24 to heat it sufficiently to bring the pressure of the oxygen passing through the barostat 16 to above the minimum setting. The controller 22 may also receive information on the amount of liquid oxygen in the Dewar 12 to prevent the controller 22 from heating the heating element 24 when the Dewar 12 is empty. The barostat 16 may be replaced with a combination of a gas valve and a gauge.

In the embodiment shown in FIG. 3 or others (e.g., oxygen or an oxygen-containing gas supplied from a tank or Dewar 12), the gas supplied from the source may be relatively cold. Thus, the apparatus may further comprise a heater configured to heat the oxygen or an oxygen-containing gas in the conduit (e.g., after pressure regulation) to a predetermined or conditioned temperature (e.g., 25-40° C., or any temperature or temperature range therein, such as 37° C.) that is comfortable for the recipients of the gas. The gas heater may comprise a resistive heater wrapped around the conduit and optionally covered with insulation, and may be used whether the gas is from a cryogenic Dewar 12 or from a set of high-pressure tanks as described herein.

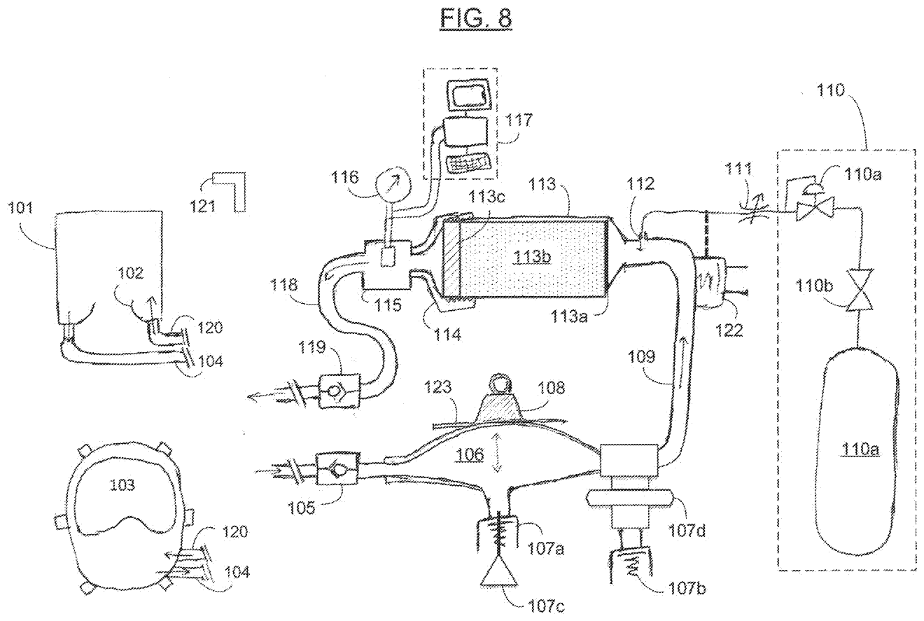

In an alternative embodiment, the Dewar 12 of liquid oxygen may be replaced by a plurality of oxygen tanks (e.g., a pallet of industrial oxygen tanks). For example, one may estimate that each patient takes in 0.5 liters of new oxygen per breath, at a rate of 12 breaths per minute. This results in each patient breathing in 6 liters of oxygen per minute. If we further assume a maximum of 200 patients per session, the aircraft's emergency oxygen system must supply a maximum of about 1200 liters of oxygen per minute. Assuming a 90-minute session, the present system should be capable of providing 12,000 liters of oxygen at STP per session, or about 12 liters of liquid oxygen at atmospheric pressure. A pallet that can hold a minimum of approximately 12,000 liters of gas at STP is suitable for an entire HBOT session of 1.5 to 2 hours for 200 patients. With the rebreather mask of FIG. 8, the oxygen consumption rate may drop by a factor of ten. To connect the tanks to the aircraft's emergency oxygen system, the tanks may be equipped with a first-stage regulator, a manifold configured to receive oxygen from the first-stage regulator and joined to a hose, tube or conduit capable of transporting oxygen at a pressure of 2.0 ATA or greater, and a second-stage regulator (e.g., in the hose, tube or conduit) to provide relatively low-pressure oxygen (but still HBO due to ambient pressure) on demand to the emergency oxygen system.

Emergency plumbing (e.g., a pump or compressor, a manifold, and conduits to each seat) is already provided in every standard commercial pressurized aircraft to provide air or oxygen to passengers in the case of a loss of cabin pressure. Such plumbing can be used to provide oxygen (HBO) from the apparatus 10 at ambient pressure inside the pressurized aircraft 1, which is designed to support a pressure differential of 0.6 to 0.8 bars, 60-80% higher than conventional ventilators at sea level. The conduit from the apparatus 10 that provides the oxygen to the airplane can be connected to the emergency plumbing (e.g., in place of the pump, compressor or a pressurized tank of air or oxygen) through an emergency air/oxygen input port 14. In some cases, the aircraft 1 may be modified to add the air/oxygen input port 14, which may be connected to the pre-existing emergency oxygen plumbing in the aircraft 1. With optional modification, validation or certification (e.g., using conduits and connections validated or certified to withstand a higher pressure), oxygen at a pressure of up to 2.0 bars or greater can be provided through the airplane's emergency air/oxygen system. Some aircraft can reach this pressure differential already, such as some models of Gulfstream aircraft, for example. In such embodiments, the conduits, valves, regulators, manifolds, connectors etc. in the HBO supply path to and in the airplane should be made of oxygen-compatible and fire-resistant materials. For example, any lubricants used in any valves, conduit connections, etc. should be perfluorinated and/or silicone lubricants.

In one embodiment, the HBO is provided to the patients (seated in the airplane seats) through the airplane's emergency air/oxygen system. In this case, bleed air from the airplane's auxiliary power unit (APU) or from a ground power unit (GPU) can pressurize the air in the aircraft cabin from the liquid oxygen in Dewar 12 or the oxygen in the oxygen tanks, thus pressurizing the emergency oxygen system. As an alternative to the APU or GPU, the aircraft engine can be used to provide power, but at a cost of an increase in fuel consumption.

In one embodiment, ground pressurization is achieved in the aircraft cabin using maintenance procedures normally used for pressurization of the cabin on the ground.

In the embodiment that provides HBO through the airplane's emergency air/oxygen system, customized oxygen masks or breathing helmets (see, e.g., FIG. 4A) can be provided to the patients to reduce the possibility of contamination and to provide a relatively high degree of patient isolation and/or comfort during the therapy.

Alternatively, such a mask, helmet or air bag may be used in commercial pressurized airline flights. In normal flight operation, airliners have cabin air that is normally filtered and virus-free (no pathogens). An elastic or hose clamp connection could connect the cabin air supply ducts provided to each individual seat overhead to a gas input hose or tube to the input port of the helmet, mask or air bag. An optional throttleable or adjustable/variable valve may locally control the airflow into the mask, helmet or air bag. The exhaust air exits from the exhaust port of the helmet, air bag or mask in flight. The exhaust air may be optionally throttled down (e.g., the flow reduced) with a throttle valve to maintain inflation of the (inflatable) helmet. Even if there is no HBO provided or (corrugated) exhaust vacuum tube (see, e.g., the discussion with regard to FIG. 4B), the helmet is still useful to maintain a positive pressure with cabin air and isolate the user from coughs, sneezes and contamination from neighbors. This isolation may include prevention from contamination of the eyes or nose while the helmet is being worn. In one embodiment, the exhaust air exits the optionally throttled exhaust port. In another embodiment, the exhaust air leaks out of the neck seal, which may be elastic, a hook-and-loop fastener (e.g., Velcro), or other sealing band.

Another advantage of the present method is that flight crew (e.g., flight attendants, maintenance crew, cleaning staff, etc.) who might otherwise be unemployed can ensure patient safety while the plane is on the ground and while the cabin is pressurized. Personal Protective Equipment (PPE) can be utilized by all attending and support personnel to reduce or eliminate further infections.

Before every HBOT session, the cabin (e.g., seats, seatbacks, armrests, floors, walls, ceiling, bathrooms, etc.) can be sanitized and/or disinfected by electrostatically spraying with alcohol (e.g., ethanol). Ideally, one 90-minute HBOT session can be provided every two hours. Furthermore, patients can receive HBOT therapy without entering the hospital. If the patient is hypoxemic, the patient can go directly to the airplane's location. Alternatively, the patient can be transported to the airplane's location from a hospital or medical clinic.

In an alternate embodiment, the aircraft is emptied (e.g., of people, waste, loose items, etc.) after use, sealed and filled with a disinfectant gas such as ozone. The ozone disinfects all of the surfaces and inactivates virus particles that may be present in the cabin. The ozone is then purged from the cabin, and the cabin is ready for use again in a matter of minutes. An ozone generator can be installed in the cabin and used only when the cabin is sealed and empty. This disinfectant gas embodiment may accelerate the turnaround speed (e.g., for preparing the cabin for the next therapy session) with less residual effects, and may improve operational performance.

In a further alternative cleaning procedure, prior to the first therapy session and/or after the last therapy session on a given day, one may spray the aircraft cabin with an electrostatically-charged alcohol, which may be applied to all services (e.g., in the cabin). In addition, between sessions, one may perform the ozone cleaning of the cabin. In a matter of minutes, the ozone (which can go anywhere inside the cabin that a gas can go) disinfects all of the cabin surfaces. Then, the cabin atmosphere is purged (e.g., with air), and new patients may board the aircraft. The latter procedure enables a very quick and easy to disinfect the cabin in a matter of minutes.

FIG. 4A shows patients in one row of the airplane cabin receiving HBOT in accordance with at least one embodiment of the present invention. The patients may wear a head/face shield or “helmet” 30a-b, receiving HBO from the tubes 38a-b that drop or extend down from the ceiling and that are connected to the emergency oxygen system of the aircraft, and exhausting exhaled gasses through exhaust tubes 32a-b. The exhaust tubes 32a-b are connected to the helmets 30a-b through connectors 34a-b. The exhaust tube connection 34a shows the outside, and exhaust tube connection 34b shows the inside (through the head/face shield 30b). The exhaust hoses 32a-b may go down to the floor of the aircraft and run along the floor to a connection port 36 (FIG. 4B) in the wall of the aircraft cabin.

The face shield/helmet 30 may be secured around the patient's neck using an elastic material (e.g., comprising rubber or another elastic material such as a conventional elastic band used for clothing) that allows for comfortable breathing but also provides a reasonable air seal around the patient's neck. This enables an appropriate pressure of oxygen inside the face shield/helmet 30, which should be at or slightly above ambient pressure (e.g., 1.0 ATA) to 2.0 ATA (or any range of values therein). The exhaust tubes 32a-b can be connected to an adjustable-pressure conduit system and manifold in the cabin wall, that can be at a lower pressure than ambient pressure (e.g., using a vacuum pump or fan). Such an apparatus can contain any particles (infectious or otherwise) expelled from a patient as a result of coughing, as shown in FIG. 4A.

In one embodiment, the face shield/helmet 30 may comprise plexiglass or a polycarbonate, similar to the helmet for flights operated by Virgin Galactic (Mojave, Calif.). However, simpler, less expensive and/or more flexible materials can be used, such as a flexible clear plastic bag (e.g., comprising polyethylene, polypropylene, low-density versions or copolymers thereof, etc.) that can inflate at a very mild positive inflation pressure or pressure differential between the interior of the helmet/shield 30 and the cabin. The pressure inside the helmet/shield 30 can be adjusted by a throttle (e.g., a variable valve) on the exhaust tube 32. By closing the throttle on the exhaust tube 32, then the helmet/shield 30 will inflate (e.g., until it is smooth and/or not crinkly), and opening the throttle on the exhaust tube 32 will cause the helmet/shield 30 to deflate or exhaust the hyperbaric gas therein. By suitable adjustment of the exhaust throttle, it is possible to adjust the pressure within the helmet/shield 30. When not in use, the flexible plastic helmet 30 can be folded into a compact size and/or shape.

In a further embodiment, a throttle can also control the input of oxygen through the tube from the ceiling into the helmet 30 (e.g., rather than on the exhaust hose 32), in which case opening the throttle increases the oxygen pressure and causes the helmet 30 to inflate around the head.

The supply hoses 38 (from the ceiling) and/or the exhaust hoses 32 can comprise vacuum hoses that may be corrugated and/or flexible (e.g., similar to those connected to the radiator of a car). As shown in FIG. 4B, the exhaust hoses 32 go from the helmets 30 to the floor of the cabin, then laterally across the cabin floor to an exhaust port connector under the window of the aircraft. The exhaust port connector in the cabin wall connects one exhaust hose 32 to a manifold that effectively joins it to a main exhaust/vacuum hose running along the length of the aircraft, under the windows and between the cabin wall and the exterior shell of the aircraft, to a second manifold at the back of the aircraft connected to an exhaust filter to remove particulates (e.g., having a pore size configured to remove >99% (or any percentage greater than 99%, such as 99.5% or 99.7%) of all particles having a diameter or size <1 μm or any other maximum diameter or size <1 μm, such as 0.4 μM. After filtration, the exhaust gas is expelled through an exhaust port at the back of the plane. Alternatively, to provide even greater isolation, each exhaust hose 32 has a unique connector in the cabin wall, which is connected to a unique tube or hose that goes to the manifold in the back of the plane.

The exhaust port opening or output flow may be adjusted to change the pressure in the cabin. Normally, the cabin pressure is controlled through the exhaust port, so a combination of controlling the flow through the exhaust port and controlling the oxygen supply pressure maintains the pressure differential (e.g., in the range 5˜25 psi, for example 9 psi) between the cabin and the atmosphere outside the plane that is possible, for example, in a Boeing 747 or 787 aircraft, and in other advanced aircraft such as the Boeing 777X and at least one Airbus aircraft in development that can provide a relatively high cabin pressure (e.g., a cabin altitude of 6,000 ft MSL, rather than 8,000 ft MSL, when flying at an altitude of 35,000-45,000 ft). Such aircraft may be optimal for delivering 0.6 bars of gauge pressure or 1.6 bars of absolute pressure of oxygen to the patients. With such helmets, exhaust hoses, and exhaust conduits and manifolds, the present system can combine removal of exhaled gas and pathogens out of the aircraft without substantially contaminating the aircraft or the external environment.

Social distancing can be practiced in the present HBOT method. For example, as shown in FIG. 4A, the middle seat in a 3-seat row can remain empty. In a 2-seat row, a maximum of 1 seat may be occupied, and in a 4-seat row, a maximum of 2 seats may be occupied, with at least 1 empty seat between occupied seats. Referring to FIG. 4B, even further social distancing can be practiced when possible. Patients may also be separated by rows. For example, if a patient is seated in seat XA, then the nearest patient may be seated at least one row and two seats away (e.g., in seat YC, where Y=the row number X+1). Such row staggering further increases patient and staff comfort and security. Seating may also be prioritized to space HBOT patients for a particular therapy session apart as much as possible. The aircraft may have from 10 to 50 rows of seats, each row may have 2 or more sections or groups of seats, and each row section or group may contain from 1 to 5 seats.

In FIG. 4B, other than the arrow pointing towards the nose/cockpit of the aircraft, the arrows signify the direction of exhaust gases, first through hoses 32a-b to the floor, then through the exhaust port connector 36 in the cabin wall, to one or more vacuum hoses (which may be larger than hoses 32a-b) and/or a manifold 18 that runs between the cabin wall and the exterior shell of the aircraft to the stern of the aircraft, to the tail of the cabin and to further manifolds to the filter and the exhaust flow port regulator, then out the exhaust port to the outside of the aircraft.

With use of larger diameter vacuum hoses 18 (i.e., having a diameter larger than that of the hoses 32a-b) between the exhaust port connector 36 and the pre-filter manifold, it is possible to effectively exhaust exhaled gases from the entire aircraft without power at a 6-to-9 psi (0.4-0.6 atm) differential (or greater or less; the invention is not limited to this range). Additional manifolds and exhaust (vacuum) hoses or conduits can exist between seats or rows of seats, and a different or additional exhaust manifold can run down the center rows of seats (either along an aisle or along middle seats in the rows) of the aircraft (e.g., when the aircraft is a wide-body jet with two aisles).

In another embodiment, the cabin is pressurized with gas to a pressure of 1.4-2.0 ATA, using the standard (i.e., not emergency) air pressurization system on commercial aircraft. Large commercial aircraft, such as those manufactured by Boeing and Airbus, can be pressurized safely up to a cabin pressure of about 1.6 ATA; some commercial business jets (e.g., manufactured by Lear) can be pressurized safely up to a cabin pressure of about 2.0 ATA or higher.

On-board entertainment systems can be used to entertain patients during HBOT. To reduce the possibility of contamination through entertainment system controls provided at individual seats, the entertainment system can be centrally or remotely controlled (e.g., by staff). Wi-Fi and cellular services can also be provided to the patients and staff, using pre-existing equipment available on most, if not all, commercially available wide-body aircraft. In embodiments using the oxygen masks/helmets, the use of electronic devices is allowable during treatment. If patients are treated with HBO through the aircraft cabin pressurization system, as a safety precaution, electronic devices may not be permissible.

As an alternative to the Dewar 12 of liquid oxygen (FIG. 3), multiple pressurized oxygen tanks can be fitted to a manifold connected to the hose supplying the HBO to the plane. A conventional regulator can ensure that a maximum gauge pressure of, for example, 14-15 psi is delivered to the supply hose/conduit. Support/maintenance staff may monitor the pressure in such tanks, and replace low-pressure tanks by closing a valve between the tank and the manifold, exchanging the low-pressure tank for a full tank, and opening the valve.

An alternative to the external HBO supply apparatus 10 is to place the HBO supply apparatus (or components thereof) in the plane's cargo hold, optionally towards the front (nose) of the aircraft.

Another alternative is to administer hyperbaric oxygen therapy in a hospital-like or an intensive care unit (ICU)-like setting on an aircraft. Such settings may be available on or in conventional medevac or casevac aircraft. Alternatively, conventional medevac or casevac aircraft may be modified to include such a setting. In at least one embodiment, one or more functional operating theaters (e.g., capable of providing “medical holiday”- or “medical tourism”-type surgical operations) may be present in the aircraft cabin, with hyperbaric oxygen provided to the patient, or hyperbaric air pressure conditions in the ICU-like setting or operating theater under some conditions. Having HBOT (as described herein) or hyperbaric pressure available in a hospital-like environment enables medical care to be provided to advanced and/or progressed COVID-19 patients who should be in an intensive care unit or hospital, but who still can benefit from hyperbaric oxygen.

With a pressurized operating theater environment, the health care providers can breathe air and operate in a relatively safe environment, and the masks, helmets or head coverings worn by the patients (which may be modified to allow for introduction of anesthesia; e.g., by controlling both the HBO and the anesthesia gas with respective valves or regulators on separate conduits that are joined together with a Y- or T-connector) provide hyperbaric oxygen to the patients without increasing the fire hazard associated with hyperbaric oxygen and without the invasiveness associated with extracorporeal membrane oxygen (ECMO) or its cost.

At least some medevac planes are pressurized and can be used or adapted for use on the ground to provide hyperbaric oxygen therapy or a hyperbaric setting. Retrofitting existing wide body (and other) aircraft, including medevac and casevac planes, to include an operating room, an intensive care unit or a hospital-like setting may produce superior therapeutic results than conventional hospital-based or -implemented therapies in a conventional hospital.

Exemplary Face/Head Coverings for Delivering Hyperbaric Oxygen

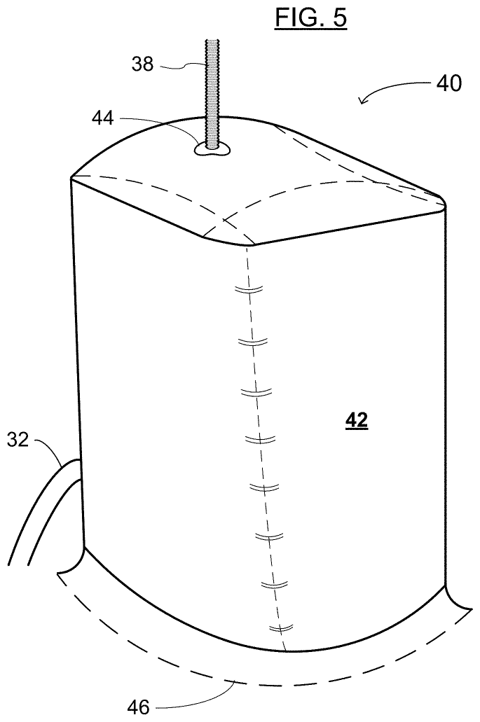

FIG. 5 shows a head covering or helmet 40 typical of those used in full body, positive-pressure cleanroom suits (e.g., for biosafety level 4 [BSL4] virology laboratories). The head covering or helmet 40 fully encloses a person's head and comprises a flexible material (e.g., an organic polymer, such as poly(methyl methacrylate) (PMMA), cellophane, or polystyrene, that is optically transparent at least in the face area 42. The hyperbaric oxygen is provided to the interior of the head covering or helmet 40 through the tube or hose 38 that drops down from the ceiling of the aircraft cabin, which is connected to the head covering or helmet 40 at a tube or hose connector 44. Gases in the head covering or helmet 40 are exhausted through the exhaust hose/tube 32, which is connected at the back of the head covering or helmet 40 using a connector similar to the connector 44.

The head covering or helmet 40 may be secured or held in place on the person via a collar 46. In some embodiments, the collar 46 comprises two layers of material, the lower or inner layer of which is oxygen-impermeable, and which may be filled at least partially with sand, a silicone gel, or other relatively safe, flexible material that adds weight to the bottom of the head covering or helmet 40 and that can form a loose seal to the person's body. In a further embodiment, the person may wear a vest or jacket designed to form a substantially airtight seal between the head covering or helmet 40 and the person's body. For example, the vest or jacket may comprise an air- or oxygen-impermeable material on at least an outer surface thereof that contacts the collar 46, and that may further include mechanisms for securing or sealing a periphery of the vest or jacket to the person (e.g., elastic bands at cuffs or around sleeves of the jacket or vest, a cinching or draw string or cord around the chest or waist of the vest or jacket secured in place with a clip or spring-loaded clamp, etc.). Alternatively, the head covering or helmet 40 and collar 46 may be integrated with the jacket or vest, similar to some commercially available personal protective equipment (PPE).

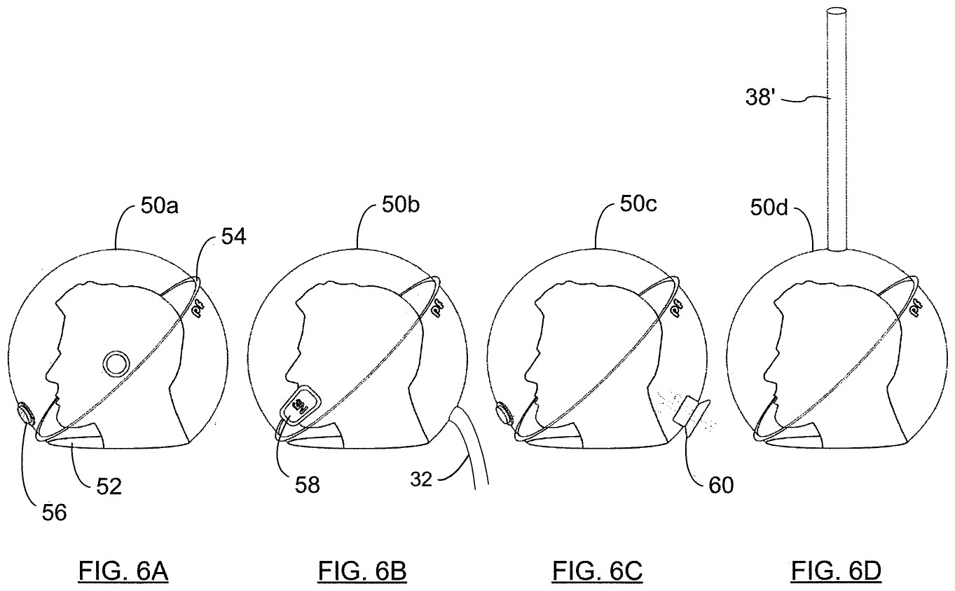

FIGS. 6A-D show alternative face, nose and mouth shields 50a-d known as iSpheres. An iSphere shield 50 comprises two transparent, hollow hemispheres that are secured together (e.g., using an adhesive and/or a screw-type or tongue-in-groove fitting), with the lower hemisphere being cut to form a hole or opening 52 through which the user inserts his or her head. The joint 54 between the two hemispheres may be at a position or angle adapted to keep it out of the person's line of sight. The iSphere is an open-source design (see, e.g., https://plastique-fantastique.de/iSphere) that can be readily downloaded. The hollow hemispheres may comprise a stiff or relatively inflexible organic polymer, such as a polycarbonate or poly(vinyl chloride) (PVC), and are generally widely commercially available.

The shield 50d (FIG. 6D) is fitted with a tube or hose 38′ (to be connected, for example, to the aircraft emergency oxygen system). The shield 50b (FIG. 6B) is fitted with a tube or hose 32 to be connected, for example, to the exhaust conduit system and/or manifold between the aircraft cabin wall and the exterior shell/fuselage of the aircraft. The shields 50a-c may be customized with accessories such as an integrated microphone 56, a speaker 58, or a cooling fan 60. The shields 50a-d may also be tinted, in whole or in part (e.g., the upper hemisphere), for example to reduce glare from internal lighting.

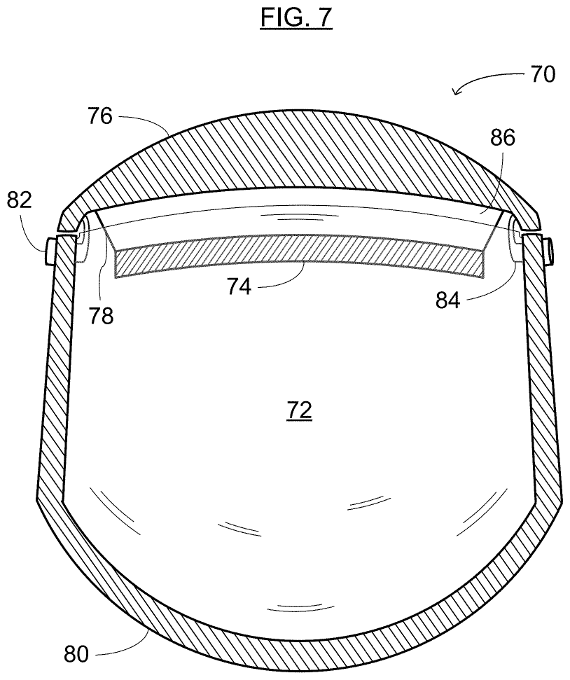

FIG. 7 shows a face shield 70, which offers more effective protection against virus infections than a relatively simple nose-and-mouth mask. For example, the shield 70 is more effective than a nose-and-mouth mask at protecting the eyes from COVID-19 infection, and may be worn by staff and medical personnel while in the aircraft, as well as by HBOT patients when receiving hyperbaric oxygen through a nose-and-mouth mask similar to those worn by pilots of high-altitude aircraft.

The face shield 70 comprises a transparent visor 72 that covers the face, plus a securing mechanism such as a strap or headband 74 to hold them in place on the person's head. The strap or headband 74 may be adjustable, and may be secured or affixed to a helmet section 76 via a frame or series of connectors 78. The visor 72 may be secured in a frame or border 80 fixed to a hinge 82. The hinge 82 has an axle or shaft (not shown) that passes through the frame or border 80. An inner surface or portion of hinge 82 (or the axle/shaft) is fixed to the helmet 76 by a brace 84. Some shields 70 are disposable, while others can be reused after sterilization.

The front edge of the helmet 76 may extend beyond the person's face by at least a few centimeters (e.g., 2-5 cm) to provide greater protection for the person's eyes. The frame 80 of the shield 70 should also extend below the person's chin in a vertical direction and to the person's ears in a horizontal direction. In some embodiments, the frame 80 may be configured or adapted to contact the person's chest (or clothing on the person's chest).

Ideally, there should be no gaps that might allow droplets to reach the person's face, although a small gap 86 between the visor 72 and the helmet 76 may exist to facilitate raising and lowering the visor 72 as needed. The face shield 70 has several advantages over nose-and-mouth face masks. They provide greater facial surface area coverage than masks, they protect all of the areas where a virus can enter the body (the eyes, nose, and mouth), a virus is unable to penetrate the polymeric visor 72 (unlike a cloth or fiber mask), and they can prevent one from touching one's face. One drawback of nose-and-mouth face masks is that many persons touch their faces to adjust the mask, which introduces a risk for infection via contaminated hands or gloves. Face shields are also relatively durable, can be cleaned after use, and reused repeatedly.

FIG. 8 shows an exemplary emergency oxygen rebreather 100 for use in accordance with one or more embodiments of the present invention. The emergency oxygen rebreather 100 creates a positive pressure and hyperbaric use of an oxygen-containing gas is a small space for use in respiratory therapy.

The rebreather 100 includes a transparent hood 101 and a replaceable neck seal or ring 102. The transparent hood 101 is commercially available from Amron International (Escondido, Calif., USA). The neck seal or ring 102 may be made (primarily) of latex or silicone. An alternative to the combination of the transparent hood 101 and the neck seal or ring 102 is a full-face mask 103. An exhalation hose 104 is connected to either the neck seal or ring 102 or the full-face mask 103, depending on the mask/hood to be worn by the patient. Similarly, an inhalation hose 120 is also connected to either the neck seal or ring 102 or the full-face mask 103, through a different port than the exhalation hose 104.

The exhalation hose 104 is connected at an opposite end to an exhalation valve 105. The exhalation valve 105 may comprise a 1-way or mushroom valve or diaphragm. The exhalation valve 105 is connected to an inlet to a breathing bag 106. The breathing bag 106 may comprise medically-acceptable and/or -approved welded polyurethane (or other medically-acceptable and/or -approved material having the same or similar mechanical properties, such as silicone and polytetrafluoroethylene [TEFLON]-coated polymers, which may have greater oxygen compatibility).

The breathing bag 106 is generally equipped with exhaust valves. For example, the breathing bag 106 may have a condensation drain valve 107a with manual purge mechanism 107c at a lower or lowermost location of the breathing bag 106. In addition, the breathing bag 106 may be connected to an automatic overpressure valve 107b at an end of the breathing bag 106 opposite from the exhalation valve 105. The overpressure valve 107b may be equipped with an optional viral filter 107d. To maintain positive pressure breathing, an optional counterweight 108 may be placed on the breathing bag 106. The counterweight 108 may have a variable weight or apply a variable force to the breathing bag 106. The counterweight 108 may be placed on a plate or tray 123. The counterweight 108 should be simple, and almost any object (such as the CO₂-absorbing canister 113) may be suitable.

A hose 109 is also connected to the overpressure valve 107b, at an end or opening opposite from the breathing bag 106. Preferably, the hose 109 is sterilizable, has a smooth bore, comprises an organic polymer such as polytetrafluoroethylene (PTFE) or other polymer having similar properties, and/or has a diameter of 20-40 mm (e.g., 22 mm).

A compressed oxygen supply 110 may be operably connected to the hose 109. The oxygen supply 110 may be as described herein. For example, the oxygen supply 110 may comprise an oxygen bottle, cylinder or tank 110a, an on-off valve 110b for the oxygen bottle, cylinder or tank 110a, and a pressure regulator 110c. In one example, the on-off valve 110b may comprise a cylinder valve. The pressure regulator 110c is conventional. The oxygen supply 110 is optional in the rebreather 100. For example, when the rebreather 100 is not in use on an airplane or other aeronautical vessel, one may use a conventional hospital O₂ supply, O₂ concentrator, liquid O₂ evaporator as described elsewhere in this document, chemical O₂ generator, or other conventional source of oxygen.

A needle valve 111 controls the flow of oxygen from the oxygen supply 110 to the hose 109. The needle valve 111 should be accessible to anyone responsible for maintaining or operating the rebreather 100. The oxygen flow from the needle valve 111 enters the hose 109 through an oxygen inlet 112.

The hose 109 is connected at an end opposite from the overpressure valve 107b to a CO₂ scrubber canister 113. The CO₂ scrubber canister 113 includes a housing that preferably comprises transparent acrylic, CO₂ absorbent material 113b, a grid 113a to retain the CO₂ absorbent material 113b, and a dust filter and grid 113c downstream from the CO₂ absorbent material 113b. In one or more embodiments, the CO₂ absorbent material 113b preferably comprises soda lime (e.g., a mixture comprising 50-90 wt % calcium oxide and 1-5 wt % sodium hydroxide), with a color-changing agent to display visually when the CO₂ absorption capacity is below a predetermined threshold (e.g., related to safety of using the rebreather 100). For example, the CO₂ scrubber canister 113 may be a standard or conventional CO₂ scrubber canister, and in one embodiment, may be an anesthetic canister. The CO₂ scrubber canister 113 may also be used as the counterweight 108. A cap 114 may be fitted to the downstream end of the CO₂ scrubber canister 113 to enable removal, opening and reloading the canister 113 with fresh CO₂ absorbent material 113b.

The rebreather 100 may further comprise an O₂ and/or CO₂ sensors 115. The CO₂ sensor increases the cost of the rebreather 100, but enables optimal use of the CO₂ absorbent material 113b, including optimal times for its regeneration, thereby reducing the logistical burden(s) associated with safe use of the rebreather 100.

The rebreather 100 may further comprise an O₂ gauge 116 and optional alarm (which may comprise the computer 117). The computer 117 as shown is linked to the O₂ gauge 116 for data logging. A pulse oximeter 121 may be used to monitor the blood oxygen level of the patient. The patient's blood oxygen levels may also be logged in the computer 117. However, data logging is not necessary in the rebreather 100.

A hose 118 is connected at one end to the O₂ and/or CO₂ sensor(s) 115 and at an opposite end to an inhalation (mushroom) valve 119. The hose 118 may be the same as or similar to the hose 109, and the inhalation valve 119 may be the same as or similar to the exhalation valve 105.

The rebreather 100 may further comprise an emergency air intake 122, operably connected to the line 109 and the oxygen supply 110 (e.g., to a line downstream from the needle valve 111). The emergency air intake 122 may comprise a conventional pressure-activated, electrically-controlled and/or diaphragm-type valve. Optionally, the emergency air intake 122 is held closed by pressure in the line from the oxygen supply 100 to the oxygen inlet 112. The emergency air intake 122 is preferably used in conjunction with the oxygen sensor 115 to avoid the risk of hypoxia. For example, when the oxygen sensor 115 detects a decrease in the pressure or partial pressure of oxygen in the rebreather 100, the computer 117 (configured to monitor the oxygen pressure as measured by the oxygen sensor 115) should sound an alarm and may send a control signal to the emergency air intake 122 to open it (e.g., to prevent a patient becoming hypoxic by means of repeatedly inhaling and recirculating air as a consequence of an insufficient oxygen supply, thus causing nitrogen to build up in the system).

The rebreather 100 comprises an extremely simple and easily constructed rebreather system to extend the available oxygen supply in an emergency situation by an order of magnitude, or enable safe oxygen treatment within air-filled hyperbaric chamber (such as the aircraft cabin, as described in one or more embodiments of the invention above). Components in the rebreather 100 may be assembled with 40 mm threads (e.g., as used in standardized NATO supplies, such as gas masks) and 22 mm pushfit connections so that it is modular or semi-modular, can be assembled on-site, and components (including optional components) can be replaced or added as necessary/desired.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

1. An apparatus, comprising:
a) an aircraft having a cabin and an emergency air or oxygen delivery system configured to deliver air or oxygen to a plurality of persons in the cabin;
b) a source of hyperbaric oxygen;
c) a pressure gauge or regulator configured to measure or regulate a pressure within said aircraft; and
d) a plurality of nasal cannulas or face or head coverings configured to provide the hyperbaric oxygen to persons in need thereof, wherein each of the face or head coverings includes a gas inlet, a gas outlet, and one or more seals adapted to contain oxygen in the face or head covering at a neutral or positive gauge pressure.

2. The apparatus of claim 1, wherein the source of hyperbaric oxygen comprises liquid oxygen in a container configured to store liquid oxygen therein.

3. The apparatus of claim 2, further comprising:
a) a heater in the container configured to add thermal energy to the liquid oxygen; and
b) a controller configured to receive a pressure of the hyperbaric oxygen from the pressure gauge or regulator and, when the pressure of the hyperbaric oxygen is below a predetermined threshold pressure, control an amount of the thermal energy added to the liquid oxygen to increase the pressure of the hyperbaric oxygen to greater than the predetermined threshold pressure.

4. The apparatus of claim 1, wherein the source of hyperbaric oxygen comprises a plurality of tanks of oxygen operably connected to the pressure gauge or regulator.

5. The apparatus of claim 1, wherein the nasal cannulas or face or head coverings comprise the face or head coverings, and each of the face or head coverings comprises a flexible, at least partially transparent head covering configured to cover an entire head of one of the patients.

6. The apparatus of claim 1, wherein the nasal cannulas or face or head coverings comprise the face or head coverings, and each of the face or head coverings comprises a stiff head covering configured to cover an entire head of one of the patients, and the stiff head covering includes an opening through which the one patient's head is inserted.

7. The apparatus of claim 1, further comprising a supply tube, hose or conduit, connected between the emergency air or oxygen delivery system and the gas inlet.

8. The apparatus of claim 1, further comprising an exhaust system, configured to remove gas(es) from the plurality of face or head coverings without releasing the gas(es) into the cabin.

9. The apparatus of claim 8, wherein the cabin has a wall, a floor and a ceiling, the aircraft has an exterior shell or fuselage, and the exhaust system comprises:
a) one or more exhaust lines and/or an exhaust manifold under the cabin floor, above the cabin ceiling, or between the cabin wall and the exterior shell or fuselage; and
b) a plurality of exhaust tubes, hoses or conduits, each connected between a unique one of the gas outlets and the cabin floor, cabin ceiling, or cabin wall.

10. The apparatus of claim 11, wherein the exhaust system further comprises a plurality of wall, floor or ceiling connectors in the cabin wall, configured to connect a corresponding one of the plurality of exhaust tubes, hoses or conduits to the plurality of exhaust lines or the exhaust manifold.

11. A kit for providing hyperbaric oxygen to a plurality of persons in a cabin of an aircraft having an emergency air or oxygen delivery system therein, comprising:
a) a pressure gauge or regulator configured to measure or regulate a pressure of the hyperbaric oxygen;
b) a conduit or conduit system configured to transport the hyperbaric oxygen from the regulator to the emergency air or oxygen delivery system;
c) a plurality of nasal cannulas or face or head coverings configured to provide the hyperbaric oxygen to the persons, wherein each of the face or head coverings includes a gas inlet, a gas outlet, and one or more seals adapted to contain oxygen in the face or head covering at a pressure greater than or equal to ambient pressure; and
d) a plurality of supply tubes or hoses, each configured to transport the hyperbaric oxygen from the emergency air or oxygen delivery system to a unique one of the gas inlets.

12. The kit of claim 11, wherein each of the face or head coverings comprises an elastic fitting or band, configured to secure the face or head covering to a face or head of one of the persons in a substantially airtight manner, and either:
a) a flexible, at least partially transparent head covering configured to cover the entire head of the one person; or
b) a stiff, spherical head covering configured to cover the entire head of the one person, the stiff, spherical head covering including an opening through which the one person's head is inserted.

13. The kit of claim 11, further comprising a plurality of exhaust tubes or hoses, each configured to transport gas(es) from a unique one of the gas outlets to an exhaust system in the aircraft.

14. An apparatus, comprising:
a) a face or head covering configured to provide hyperbaric air or oxygen to a patient in need thereof, the face or head covering including a gas inlet, a gas outlet, and one or more seals adapted to contain the air or oxygen in the face or head covering at a pressure greater than or equal to ambient pressure;
b) a breathing bag operably equipped with an automatic overpressure valve configured to allow gas to escape from the breathing bag when a pressure in the breathing bag exceeds a predetermined threshold;
c) a CO₂ scrubber canister configured to remove CO₂ from air or oxygen in the apparatus;
d) a hose connecting the CO₂ scrubber canister and the breathing bag; and
e) an oxygen supply operably connected to the hose, configured to provide the hyperbaric air or oxygen.

15. The apparatus of claim 14, further comprising (i) a first one-way valve between the gas outlet and the breathing bag, and (ii) a second one-way valve between the CO₂ scrubber canister and the gas inlet.

16. The apparatus of claim 14, wherein the face or head covering comprises (i) a flexible, at least partially transparent head covering configured to cover the entire head of the patient and (ii) a replaceable latex or silicone neck seal or ring.

17. The apparatus of claim 14, wherein the CO₂ scrubber canister comprises:
a) a housing,
b) CO₂ absorbent material within the housing,
c) a grid upstream from the CO₂ absorbent material, configured to retain the CO₂ absorbent material in the housing, and
d) a dust filter and grid downstream from the CO₂ absorbent material.

18. The apparatus of claim 14, wherein the oxygen supply comprises:
a) an oxygen bottle, cylinder or tank,
b) an on-off valve configured to open and close the oxygen bottle, cylinder or tank, and
c) a regulator configured to control a flow of oxygen from the oxygen bottle, cylinder or tank.

19. The apparatus of claim 18, further comprising a needle valve configured to control the flow of the oxygen from the oxygen supply to the hose.

20. The apparatus of claim 14, wherein the breathing bag further comprises a condensation drain valve configured to remove liquid from the breathing bag.
